(19) United States
(12) Patent Application Publication
(10) Pub. No.: US 2022/0328180 A1
(43) Pub. Date: Oct. 13, 2022

(54) PATIENT FALL MONITORING SYSTEM

(71) Applicants: Gustavo CHELLES, Sao Paulo (BR); Conrado Leite DE VITOR, Chacara Pavoeiro (BR); Romy HAYASHI, Sao Paulo (BR); Paula Renta Cerdeira GOMEZ, Sao Paulo (BR)

(72) Inventors: Gustavo CHELLES, Sao Paulo (BR); Conrado Leite DE VITOR, Chacara Pavoeiro (BR); Romy HAYASHI, Sao Paulo (BR); Paula Renta Cerdeira GOMEZ, Sao Paulo (BR)

(21) Appl. No.: 17/226,796

(22) Filed: Apr. 9, 2021

(51) Int. Cl. G16H 40/67; G16H 40/20; G16H 50/30

(52) U.S. Cl. CPC G16H 40/67; G16H 40/20; G16H 50/30

(57) ABSTRACT

A system for monitoring people's movements, based on artificial intelligence that is a cloud-based software processing three dimensional motions, includes multiple sensors displayed in a mesh network and detecting multiple variables, a radar module that processes the received data and transforms it into a risk level gradient. The risk level gradient is available to the hospital's management system and able to be combined with the patient's record. The risk level gradient is displayed and/or made available through panels, PCs, tablets, cellular phones and other communications media. The components of the system may be interconnected either by WIFI, Bluetooth, by wire or otherwise.

### BACKGROUND OF THE INVENTION

### Field of the Invention

The present invention generally relates to a method and system for monitoring people's movement and more particularly to a method and system for monitoring a patient's movement using artificial intelligence.

### Description of the Related Art

In the USA, Healthcare Acquired Infections (HAIs) generate nearly $20 billion in unforeseen expenses each year, covered by insurance companies or by the patients' families themselves. Adding to the additional costs generated by falls within health services, this figure reaches 55 billion dollars.

### SUMMARY OF THE INVENTION

The present invention consists of a systemic solution for health services that uses software algorithms with artificial intelligence to process risk data from behavior sensors equipped with Internet of Things (TOT) communication in a mesh network. The processing of information and translation into degrees of risk allows related complications, infections and falls not only to be solved as done in traditional systems but anticipated in a way to avoid an increase in patient permanence, costs, suffering and deaths, as well as reduction of revenue for shareholders.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The foregoing and other exemplary purposes, aspects and advantages will be better understood from the following detailed description of an exemplary embodiment of the invention with reference to the drawings, in which:

FIG. 1 illustrates the system of the present invention;

FIG. 2 illustrates the basic functioning of the system;

FIGS. 3 and 4 illustrate the environment seen by a main sensor of the system;

FIG. 5 illustrates how the radar sees the environment and a person in the present system;

FIG. 6 illustrates the risk panel in a patient's room;

FIG. 7 illustrates the basic system architecture;

FIG. 8 illustrates an overview of the system installed in a hospital;

FIGS. 9-13 illustrate the main physical modules of the present system;

FIG. 14 illustrates a dashboard of the present system;

FIG. 15 illustrates a cloud server of the present system;

FIG. 16 illustrates a wearable home sensor of the present system;

FIG. 17 illustrates a radar senor that can be installed in a patient's home; and

FIG. 18 illustrates the integration of basis sensors in the present system.

### DETAILED DESCRIPTION OF EXEMPLARY EMBODIMENTS OF THE INVENTION

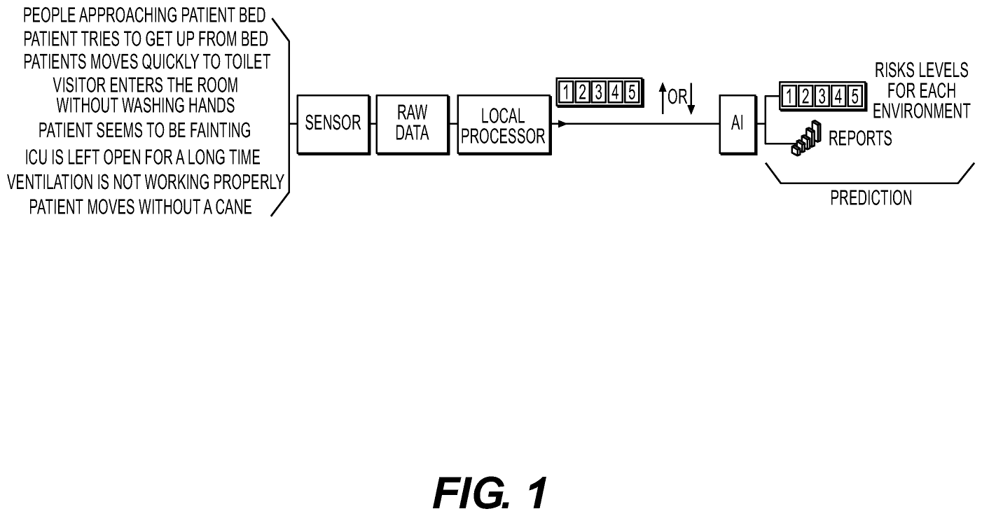

Referring now to the drawings, and more particularly to FIGS. 1-18, there are shown exemplary embodiments of the method and structures according to the present invention.

FIG. 1 illustrates the basic system of the present invention. A basic objective of the system is transforming behavioral data inside health environments into high-level risk information, capable of providing the prediction of infections and falls, so that they can be prevented, and a reaction can be implemented in a fast way. The availability of real-time data for both management and those responsible for the operational areas offers a tool for immediate reaction. Using machine learning, the system can also assist in recommending future protocols.

The system's prediction and prevention capability are implemented through cloud-based artificial intelligence and management software, which processes three-dimensional behavioral motion monitoring data in the healthcare environment, acquired by various types of behavioral sensors that can be added to build a mesh network. Each sensor is responsible for monitoring variables such as movement, speed and constancy of movement, use of protective equipment or assisting items for walking, positive pressure inside a room, opening act and opening time of doors, ventilation, number of people in the room and other parameters. The sensor data are processed locally in the radar module and transformed into a risk level gradient, increasing, reducing or maintaining a pre-existing risk level for each patient in the monitored environment. Local processing for risk gradients aims to reduce the transmission of raw data, considering that communication systems will not have a broad bandwidth and there's no need to allocate cloud space for raw data storage.

The system does not store raw data, capture images or store any personal data of patients, only an environment code and the previous level of risk of the person who occupies it. The hospital's management system uses the environment code associated with the risk information to interconnect with the patient's record, dispensing the system's need to have the personal database of patients in its cloud.

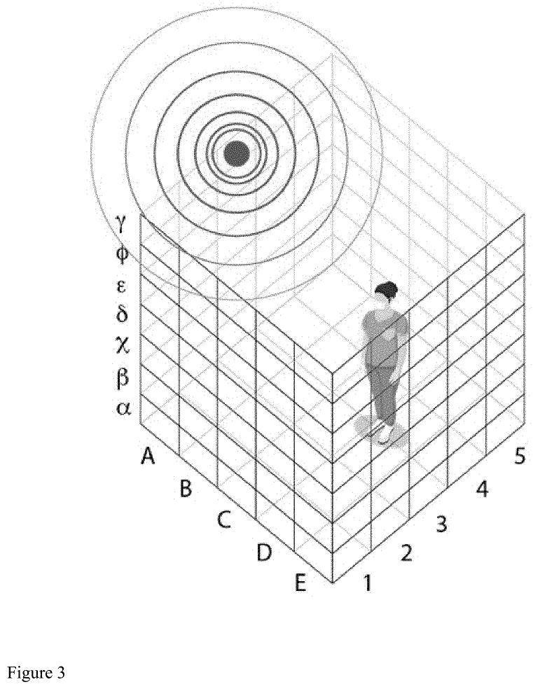

FIG. 2 illustrates the basic functioning of the system, where sensors send data to the cloud about increasing, maintaining or reducing risk levels (represented here from 1 to 5) to the software that runs in the cloud. This software, in addition to assessing the risks for making predictions, supplies dashboards, risk panels and alarms. The use of mesh networks for the sensors improves the communication reliability and allows the removal or addition of sensors without affecting the functionality of the system as a whole.

A main sensor of the system uses a type of radar that emits, receives back and processes high-frequency electromagnetic waves that can even penetrate some kinds of objects and furniture, in order to be able to map an environment three-dimensionally, from acquiring reflected waves in objects. By processing this information locally and sending filtered data to the algorithm hosted in the cloud, it is able to calculate the number of people inside it, their movements and the coordinates of the geometric center of each one over time. The radar has the advantage of not generating images that could identify people, but only a misshapen point cloud, preserving privacy and ensuring anonymity. The module has no lens, just a blind panel, which brings more comfort and prevents the embarrassment of patients and professionals. The aim of the system is not identification or punishment, but prediction and prevention, as well as the improvement of protocols.

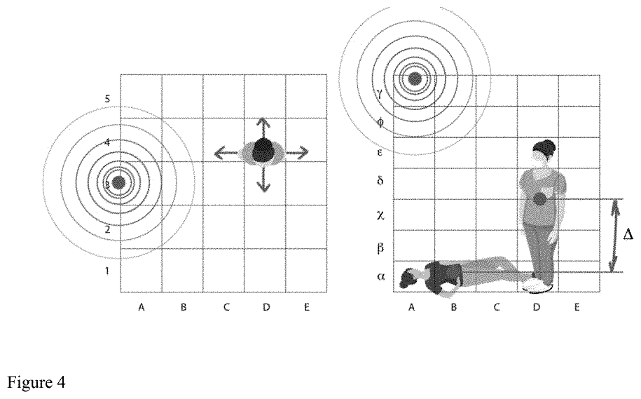

FIG. 3 illustrates that the system's main sensor sees an environment in a three-dimensional way and maps it to provide X/Y/Z coordinates of objects and people, tracking their location and movement over time.

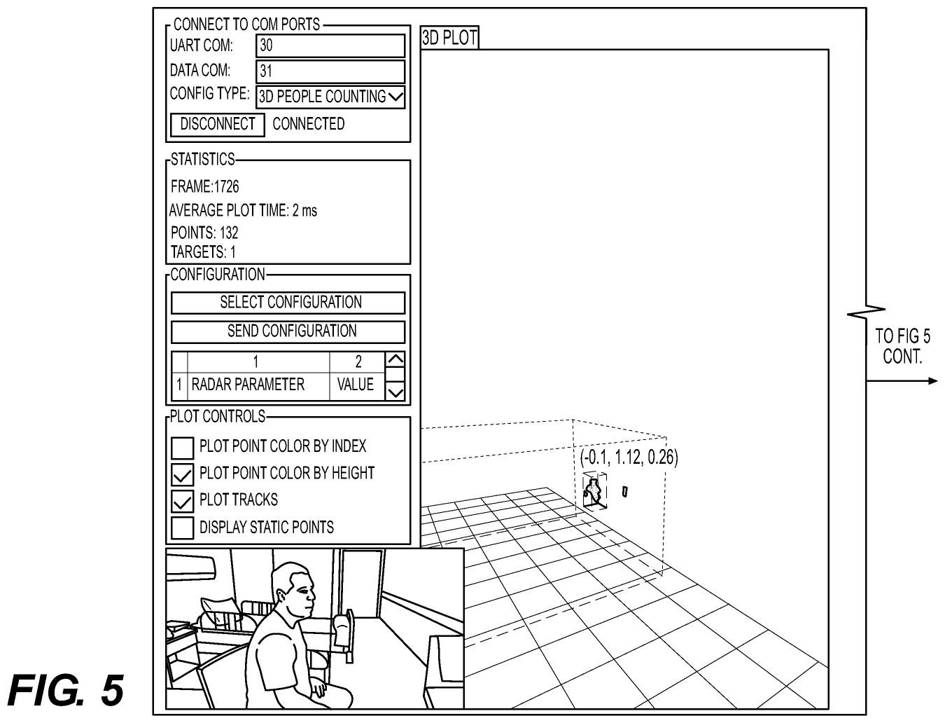

As illustrated in FIG. 4, the main sensor information can either define location on the floor plan, or measure changes in the height of people's geometric center in relation to the floor, allowing the assessment of falls. They can also detect the number of people in the environment.

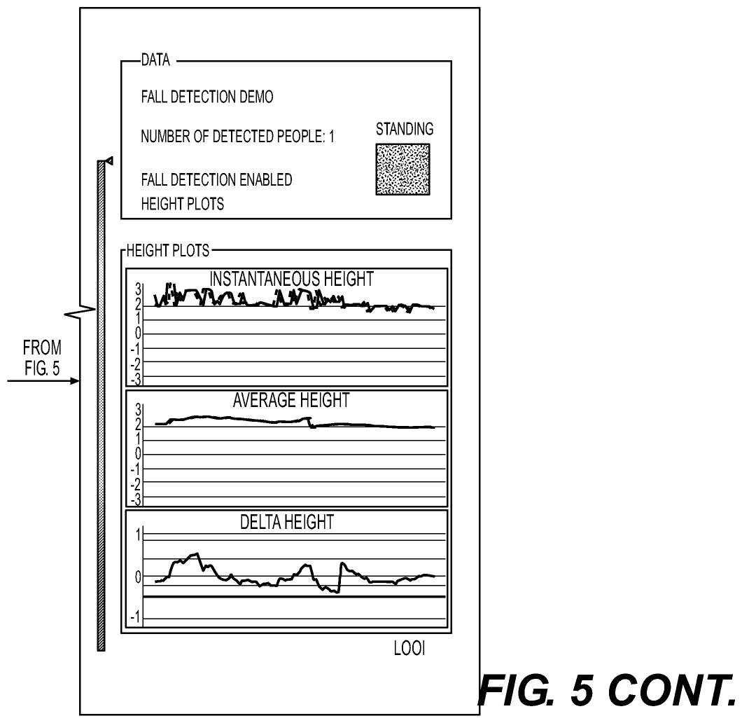

FIG. 5 illustrates how the radar sees the environment and a person in the present system in a proof-of-concept prototype—blue points cloud on the black board, where red and blue lines represent the room in three dimensions. On the right, the demonstration of how the sensor understands the center of mass of a body, its distance from the ground, in order to assess whether or not there is a fall happening.

The association of the three-dimensional sensor with other sensors, such as presence, precise movement wearable sensor, door opening and air pressure, can provide valuable information on the following parameters:
Proximity between people and the patient, including act of touching.
Hand hygiene before and after seeing the patient.
Changing the cap, mask, gloves and other safety items before and after seeing the patient.
Pressure difference between environments, in order to promote biosafety.
Patient movement, speed, pattern of behavior, transit between environments and length of stay.
Use of supports for movement, such as walking cane and walkers.

With the algorithmic processing of these parameters, we are able to provide the following service. Integration of all information obtained by the sensors with 100% privacy, using artificial intelligence in order to generate instantaneous and historical degrees of risk through dashboards and reports, which can be numbers, colors or other signs, that facilitate the understanding of how much the environment, or the patient are subject to infections, falls and other outcomes that can lead to postponement of discharge, worsening and eventually death. Based on these degrees of risk, management measures and protocols can be established before the situation occurs or worsens, because in terms of infections or falls, the reaction time is critical.

The present system makes predictions by risk analysis using artificial intelligence and machine learning. High levels of risk in environments (cells) indicate that the occurrences of infection will increase, according to rates that are calculated by historical averages and that continuously supply the machine learning system, becoming part of it. Prevention is a consequence of prediction and allows reaction in time to prevent the problem from escalating.

The degrees of risk are calculated from variables measured by the sensors, such as:
Approaching people to the bed, with or without hand hygiene—the more people approach the bed, the greater the risk gradient.
Touch the patient—increased risk. Behavior, time and number of visitors—the more visits and length of stay, the greater the risk of infection.
Passage of people through certain areas, including compliance with cleaning protocols—if the people responsible for cleaning do not go through the entire circuit spending the necessary time, the system reports a gradient increasing the risk.
Access to dispensers of sanitizers and protective consumables, such as gloves, cloaks and masks, which need to be changed constantly according to COVID-19 protocols—failure to comply with protocols generates an increase of risk.
Time using the bathroom—a long time might indicate a fall or faint in the bathroom, if there is no sensor inside the bathroom.
Environmental variables, such as air flow, pressure, temperature—absence of positive pressure in procedure rooms, caused by a door open for too long, generate increased risk.

Risks are a function of system variables in association with each patient's condition, which we call prior risk. Infection risk scan take 5 levels, for example:

1. Very Low

2. Low

3. Medium

4. High

5. Very High

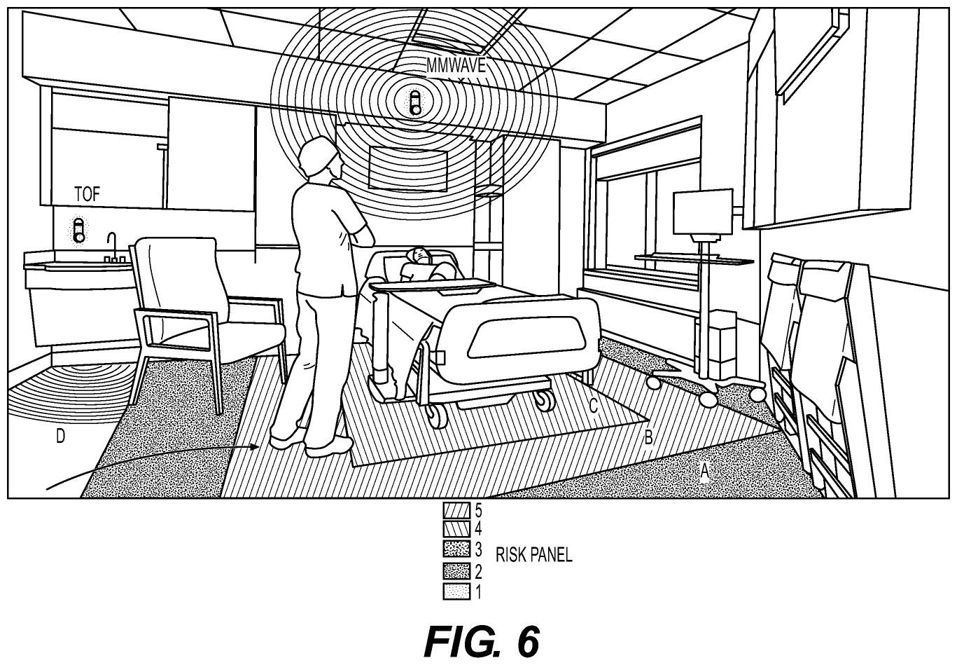

Hypothetically, a patient undergoing chemotherapy who has a depressed immune system, could start at the cell at risk 3. If a nurse enters the cell and approaches the patient's bed without hand hygiene, which is detected by the system through two sensors (radar and presence), the final risk level in the cell rises to 5, which generates an alarm at the local risk panel, at the nursing station and also in the system reports LOG (as illustrated in FIG. 6). If that same nurse, instead of approaching the bed, had only reached a distance of 2 meters, the final risk level would rise to 4. The same could happen if the patient was not so immune-depressed, not reaching the maximum level risk.

FIG. 6 illustrates how the system sees a monitoring cell with two sensors, one with three-dimensional space and the other with presence to validate hand hygiene—the system maps zones and monitors who moves and how they move, as well as the number of people in each zone. If, for example, a nurse enters the zone closest to the patient (C or B) without going through zone D, where he must clean his hands, the risk panel shows and increased grade by one or two points, depending on the patient's previous risk.

The behavior that raises risk in the cell does not guarantee that there will be infection, it only increases its risk, which for statistical reasons will become infection in a certain number of cases in the near future according to numeric trends. If a user knows about the increased risk and correlation with the complications, it is possible to infer this conversion and prevent it from becoming a reality in a broad way in the health environment, thus changing the future.

The service of prediction, prevention and reaction to falls is based on a combination of behavioral/movement monitoring and a previous risk rate, related to the patient's characteristics and condition.

The present system can be used to monitor movements of the following types:
Movement intention
Intention to get out of bed
Seizures on bed
Repetitive movements
Decubitus change
Position changes to bedsore prevention
Risky movements
Abrupt or aggressive movements
Wobbly movements
No use of supports for mobility, such as a cane and walker
Nursing presence for compliance with protocols

The previous degree of risk in this case is determined based on information from the patient's record, such as:
Age
Type of illness or physical limitation
Ability or dexterity of locomotion
Influence of medications on locomotion
Level of anxiety or behavioral characteristics

The integration between previous risk level and movement monitoring provides the final risk level showing on the risk panel and on the management dashboards. A high prior risk can lead to a high final risk. A low previous risk and unsteady movements increase the final degree of risk to the point of alarm at the nursing station. The degrees of risk can be:

1. Very Low

2. Low

3. Medium

4. High

5. Very High

The system can be configured to enter “Attention mode” when the risk assumes values above average, triggering attention alarms for the nursing station and pre-recorded messages for the patient:
“Please move more slowly”
“Please move around more calmly”
“You are not allowed to get out of bed alone, please return and call the nurse”
“If you are having problems, please call the nurse”
“Don't forget to use your cane or walker”

In the event of a fall detected by the system, an emergency alarm is triggered at the nursing station and the following messages can be triggered for the patient, depending on their previous risk:
“Stay calm and wait for help”
“Keep your current position, try to relax and don't try to get up alone, as help is on the way.”

The basic architecture of the present system is based on software and hardware, being an open system. That is, it can receive complementary modules in the future, including from other manufacturers, in addition to integrating with existing or future hospital systems.

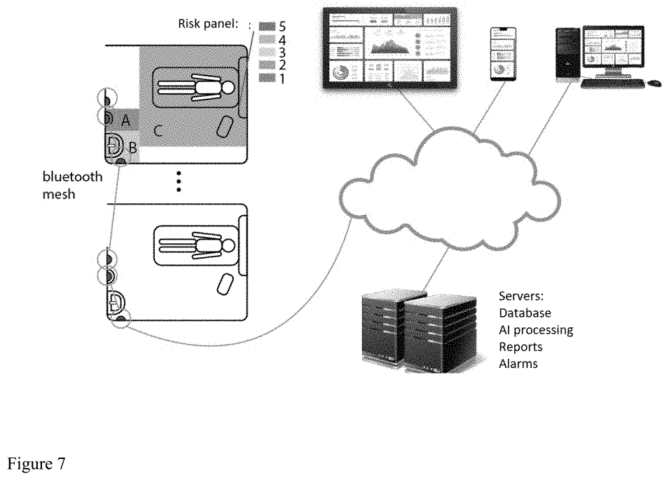

FIG. 7 illustrates the basic system architecture, showing the monitored cell, its sensors and the risk panel, which can be in the cell or on dashboard panels in nursing stations and in the cloud. Cloud servers process information from sensors to supply dashboards. The sensors are interconnected by Bluetooth Mesh protocols, which allows scalability without the need for signal coverage in all locations, and automatic reconnection to the next node if one of them stops working.

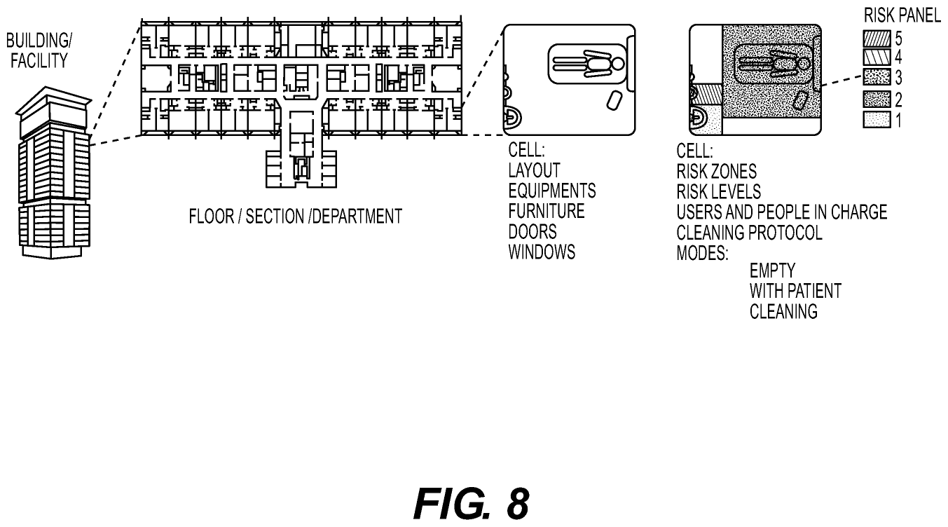

FIG. 8 illustrates an overview of the installation in a hospital, showing on the right the mapping of zones in a cell. The present management system will bring a module for configuring plants for graphical display of cells and construction of room layouts, including positioning of beds and equipment.

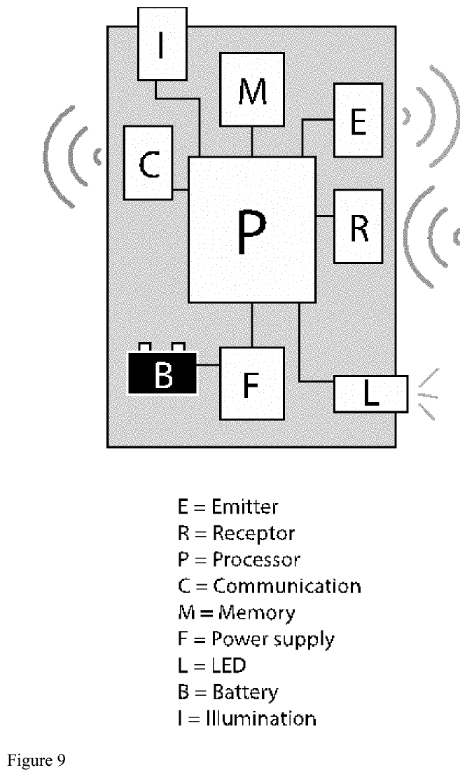

The main physical modules of the present system are described with respect to FIGS. 9-13.

FIG. 9 illustrates the main sensor. This is the base sensor of the system, because its CPU performs basic processing of the information of the cells so that the data traffic is reduced in the Mesh network—allows us to pass data of high level, instead of raw data or RAW. It controls the radar system, capable of three-dimensionally mapping environments, tracking movements and counting people. It can be equipped with a lighting sensor, to turn on the bedroom lights if the patient makes the movement to get up, which would require an integration of the lighting system to the network, with TOT lamps.

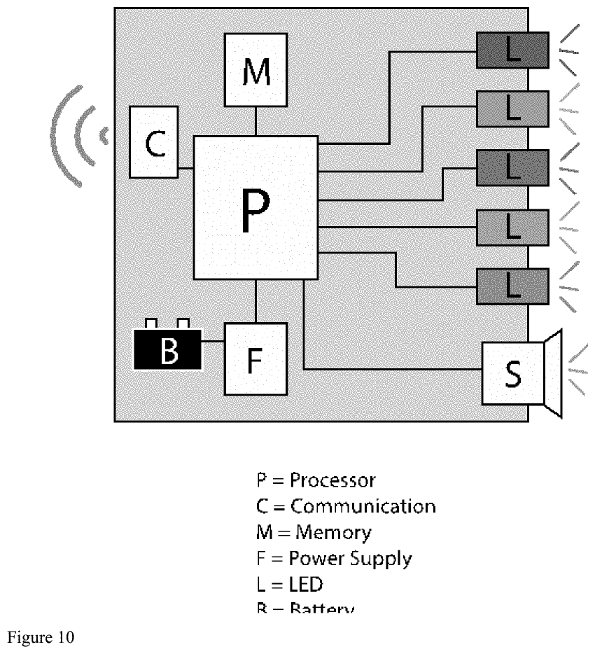

FIG. 10 illustrates the RiskPanel, which is a panel that informs instantaneous risk levels for immediate reaction, and the degree of risk can be defined both by the Radar Sensor and by the system servers in the cloud, transmitted by the panel. The degrees of risk can be associated with LED colors, such as:

1. Very Low—Green

2. Low—Blue

3. Medium—Lilac

4. High—Orange

5. Very High—Red

If the system detects that the risk of falling is high, guidance messages are passed on to the patient, such as: “Walk more slowly to avoid falling” or “Use the walker, given your condition.”

In case of a fall, the messages can be: “Keep calm and stay on the ground, someone is coming to help you.”

In the event of protocols breach, such as getting close to the patient without hand hygiene, the system can send messages such as: “Please don't forget to wash your hands” or “Please don't forget to follow protection protocols.”

Custom messages can be configured on the system. There may be an array of LEDs for risks of infection and another for risks of falling, or some form of switching between the two.

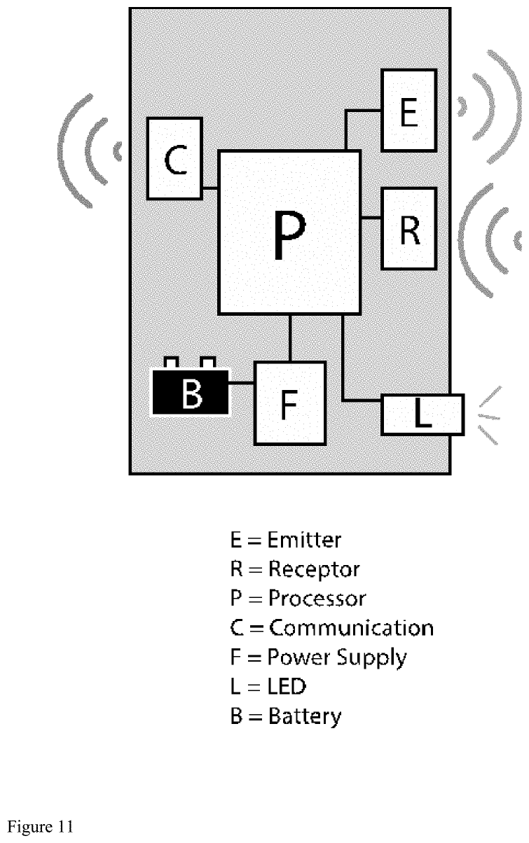

FIG. 11 illustrates a presence sensor, which is a short-distance sensor for detecting simple procedures such as hand hygiene, time to clean, access to the glove box and cloaks, among others. It communicates with the radar type sensor to send data, which will assist in the risk calculation algorithms.

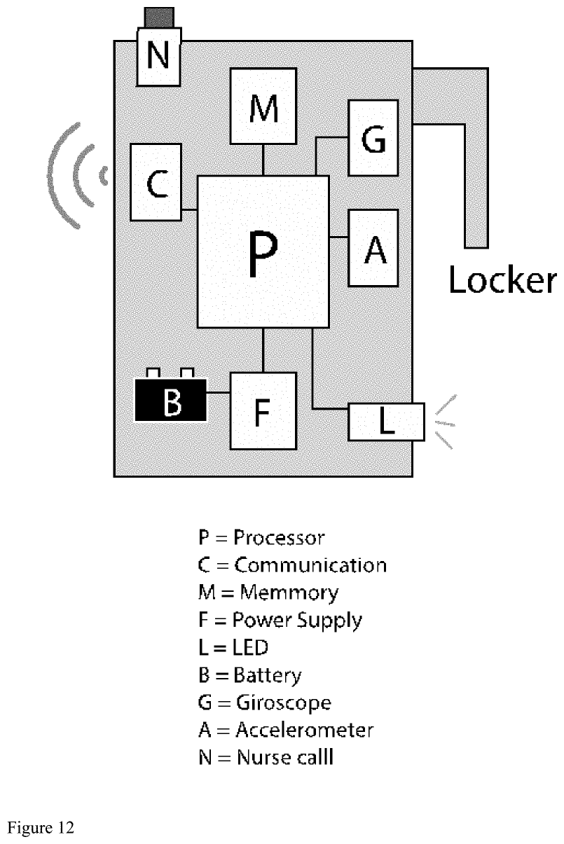

FIG. 12 illustrates a wearable precise movement sensor, which increases the accuracy of monitoring the patient's precise movements, gathering information from the accelerometer and gyroscope, such as:
Fall
Wobbly movements
Sudden movements
Decubitus and lying movements, useful to assess risks of bed sores in the ICU
Lack of movement
Seizures or some types of strokes

It can be attached to the skin, wrist, neck or attached to clothing. It can be configured to work outside the hospital environment, with connection to a smartphone or router for remote monitoring of the patient when he is at home. The sensor must be waterproof to be used in the shower or bath. It may have a version with a cardiac monitor, thermometer and oximeter, which can contribute to the risk assessment. It has an emergency nurse call button, which uses the trigger module.

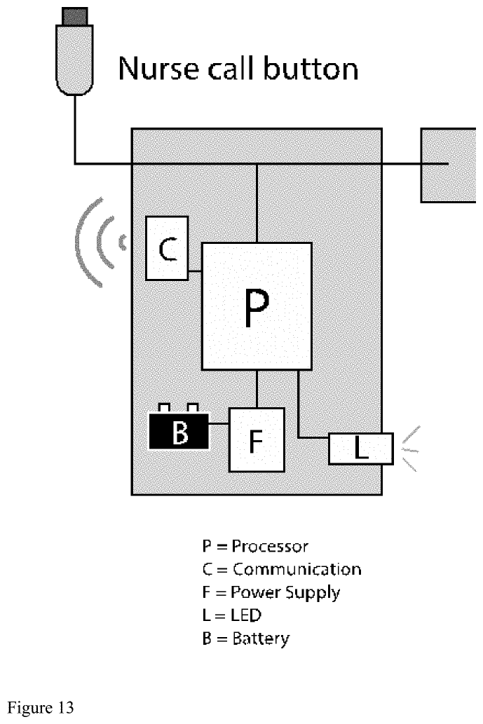

FIG. 13 illustrates a nurse calling trigger, which works in conjunction with the wearable sensor. It can be connected between the nursing call button and its door on the wall, sending an emergency signal in case of a fall. The wearable sensor and driver assembly can work independently from the present system, being a cheaper modular solution for certain applications.

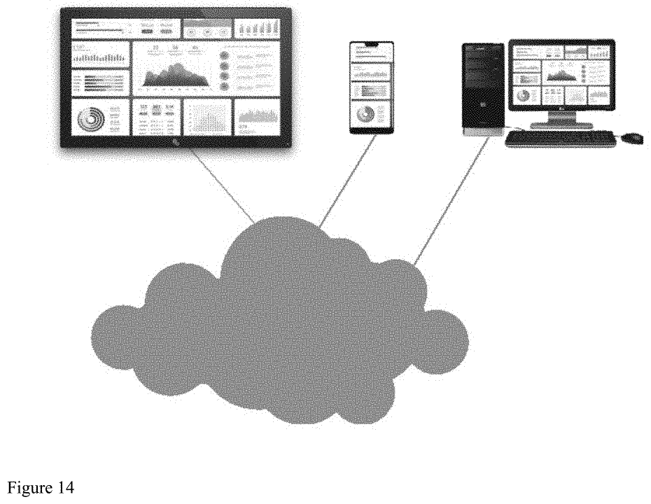

FIG. 14 illustrates a dashboard of the present system. The dashboard includes visual panels with instant or historical information graphics, allowing the visualization of the risks of the cells of a floor or of a department. Dashboards can be displayed on screens at the nurses' station, on mobile devices, or on PC's, being based on cloud applications.

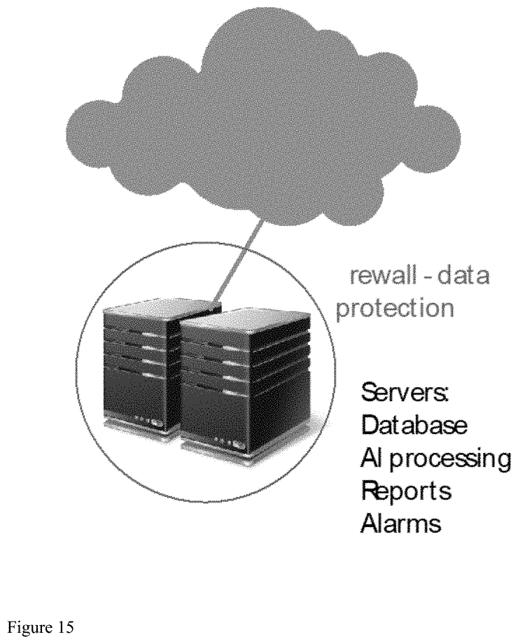

FIG. 15 illustrates a cloud server of the present system. Cloud-based servers, hosted in trusted, redundant structures, that comply with data protection laws and are safe from attacks. The servers will run the risk data analysis algorithms for each cell and build prediction and early reaction reports. They will be able to cross-check this information with patient permanence data beyond the expected time, for continuous improvement of the system using machine learning.

The system may include additional sensors, which can be of several types, such as:
Pressure difference between rooms (for infection prevention)
Use of walkers or crutches, placing a proximity flag on them that matches the wearable sensor (to prevent falls)
Access control (to avoid patient transit through certain areas that offer more danger)
Airflow (for correlation with infections)
Opening windows (for correlation with infections)
Temperature (for correlation with infection rates)
Opening doors (for operating rooms or procedure rooms)
Open door time (for surgery rooms or procedure rooms)
Lighting (to assess the degree of risk in moving the patient with lights off in the room, and eventually switching on IOT lamps)
Concentration of molecules of a certain type in the environment, such as ions, droplets and dust (for patients with hypersensitivity and surgery or procedure rooms).

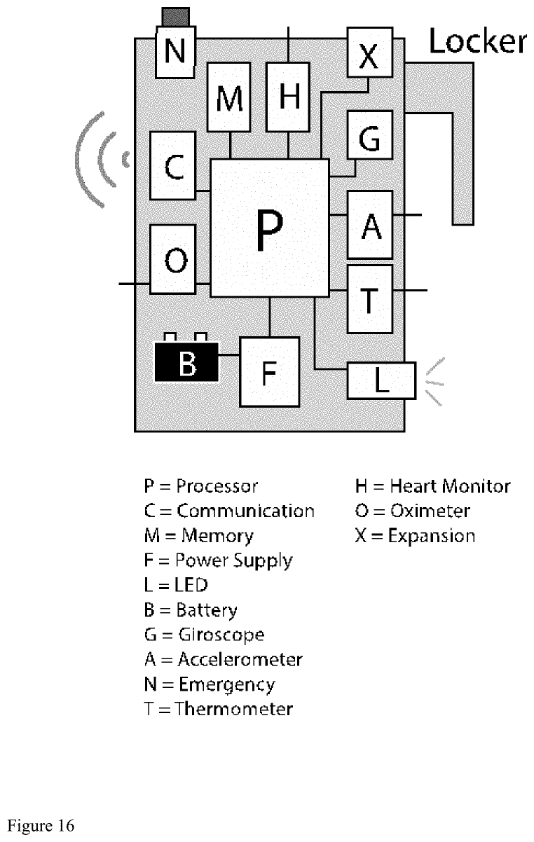

FIG. 16 illustrates a wearable home sensor of the present system. The wearable sensor is connected to the present system via the internet or a smartphone, with the ability to monitor:
Fall
Wobbly movements
Sudden movements
Decubitus and lying movements
Lack of movement
Seizures or some types of strokes
Oxygenation
Heart rate
Temperature
Electrocardiogram (with add-on)
Electroencephalogram (with add-on)
Polysomnography (with add-on)
Description of symptoms and message exchange with application on Smartphone

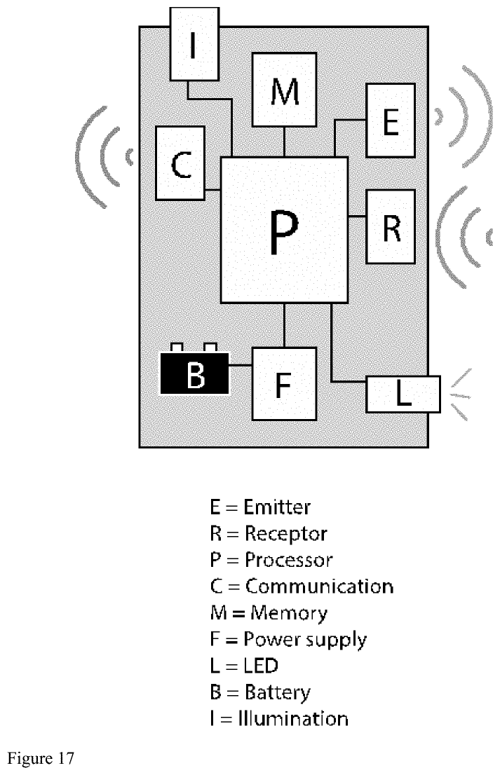

FIG. 17 illustrates a radar senor that can be installed in a patient's home. If infection control is also critical at home, a radar sensor can also be installed in the patient's room, communicating with the wearable sensor.

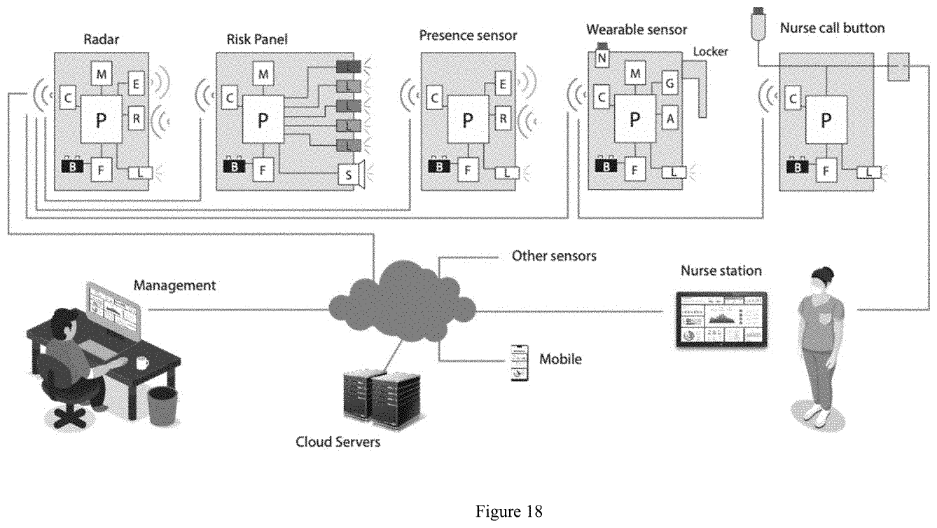

FIG. 18 illustrates the integration of basis sensors in the present system. Integration of basic sensors forming the open network of the system. New sensors can be included without limit, and the construction of a Mesh network makes devices exchange messages with each other without the need for a hub—each device is a messenger that passes messages from another, forming a line that can be extended with just an internet access point. In this type of network, if one device is eliminated, another assumes its function as a messenger so that communication is not broken.

The present system provides numerous advantages over conventional systems. That is, currently existing systems are reactive and preventive, not predictive. Combination of pre-existing technologies for an innovative system, with great potential to reduce billionaire costs currently existing in health systems. Lack of recording images, ensuring the privacy of patients and health professionals, allowing the system to be installed in rooms and ICUs, where people can be naked and fragile. Scalability of modules and cells—infinitely scalable open system. Exponentiality. Plug and play technology, which does not require investment in new infrastructure in addition to the existence of sockets and Internet signal, allows sales and remote configurations, accessing markets without the need for physical presence. Use of artificial intelligence to analyze massive data, in a way that humans would not be able to do in real time. Continuously increasing knowledge production with machine learning, which accredits operators to consultancy services in reducing infections and falls. Application of the solution architecture in other problems, such security of artwork in Museums, observation of POS behavior, industrial security, etc. Frequency of operation close to that of 5G (but not conflicting), operating in a range that has already opened paths for certification and tests of coexistence with the existing technology, reducing the risk of interference with hospital equipment.

While the invention has been described in terms of several exemplary embodiments, those skilled in the art will recognize that the invention can be practiced with modification within the spirit and scope of the appended claims.

We claim:

1. A system for monitoring a person's movement, based on artificial intelligence, wherein the artificial intelligence is a cloud-based software that processes three-dimensional behavioral motion monitoring data, the system comprises:
multiple of sensors displayed in a mesh network, said multiple sensors detecting variables including movement, speed of movement, constancy of movement, use of protective equipment or assisting items for walking, positive pressure inside the room, opening act and opening time of doors, ventilation, number of people in the room and access of each person to a particular place in the room;
a radar module configured to process received data and transform the received data into a risk level gradient, increasing, reducing or maintaining a pre-existing risk level for each patient or person in a monitored environment,
wherein the risk level gradient is made available to a hospital's management system and being combinable with a patient's record,
wherein the risk level gradient is displayed and/or made available through panels, PCs, tablets, cellular phones and other communications media, so that hospital staff may react to events including the patient falling, and
wherein components of the system are interconnected by WIFI, Bluetooth, and/or wire.

2. The system of claim 1, wherein one of the multiple sensors is a radar sensor that emits, receives back and processes high frequency electromagnetic waves that can penetrate some types of objects and furniture for three dimensionally mapping the environment through bounced back waves,
wherein the radar module is configured to:
process information from the one sensor and communicating it to a cloud; and
calculate the number of people in the room, their movements and the coordinates of the geometric centers of each person over time.
